(19) United States
(12) Patent Application Publication
(10) Pub. No.: US 2024/0041589 A1
(43) Pub. Date: Feb. 8, 2024

(54) LENGTH-ADJUSTABLE OSSICULAR PROSTHESIS HAVING IN-SITU ELONGATION OUT OF THE HEAD PLATE

(71) Applicant: Heinz Kurz GmbH, Dusslingen (DE)

(72) Inventors: Alexander SANDER, Titisee-Neustadt (DE); Thomas STIEGELE, Riederich (DE); Matthias MERTENS, Kaltenkirchen (DE); Harald HAAS, Moessingen (DE)

(73) Assignee: Heinz Kurz GmbH, Dusslingen (DE)

(21) Appl. No.: 18/225,291

(22) Filed: Jul. 24, 2023

(30) Foreign Application Priority Data
Aug. 3, 2022 (DE) 20 2022 104 408.2

(51) Int. Cl. A61F 2/18

(52) U.S. Cl. CPC A61F 2/18; A61F 2220/0033; A61F 2002/183

(57) ABSTRACT

An ossicular prosthesis has first and second fastening elements, a connecting element and rib elements. The rib elements lead directly into coupling regions of the first fastening element within the head plate plane and are movably connected thereto. All rib elements are movably connected to a coupling element and are firmly but detachably connected during operation, the coupling element in turn being rigidly connected at the other end to either the first or the second fastening element. The rib elements form the connecting element with the coupling element and are spread sectionally radially from the longitudinal axis in the middle ear in situ with the introduction of a force parallel to the longitudinal axis and thus shorten the axial functional length of the prosthesis, but increase it with the introduction of a force anti-parallel thereto. The rib elements retain, in situ, their adjusted radial position when no force acts.

### CROSS REFERENCE TO RELATED APPLICATIONS

Applicant claims priority under 35 U.S.C. § 119 of German Application No. 20 2022 104 408.2 filed Aug. 3, 2022, the disclosure of which is incorporated by reference.

### BACKGROUND OF THE INVENTION

### 1. Field of the Invention

The invention relates to an ossicular prosthesis which replaces or bridges at least one member or parts of a member of the ossicular chain, the ossicular prosthesis comprising, at one end thereof, a first fastening element designed as a head plate for mechanical contact with the tympanic membrane and/or with the handle of malleus, and, at its other end, a second fastening element for mechanical connection to a member or parts of a member of the ossicular chain or to the inner ear, and a connecting element which connects the two fastening elements to one another in a sound-conducting manner along a longitudinal axis, and the connecting element having rib elements which -at least sectionally- can be spread to a greater or lesser extent radially outwards away from the longitudinal axis, and thereby shorten the axial length of the ossicular prosthesis to a greater or lesser extent.

### 2. The Prior Art

Such a device is known from U.S. Pat. No. 10,687,937 B2 (= reference [1]).

Ossicular prostheses are used to transfer the sound from the tympanic membrane to the inner ear in the case of completely or partially missing or damaged ossicles of the human middle ear. In this case, the ossicular prosthesis has two ends, depending on the specific circumstances one end of the ossicular prosthesis resting, for example by means of a head plate, on the tympanic membrane or the handle of malleus, and the other end of the ossicular prosthesis being fastened, for example, to the stirrup bone of the human ossicular chain or inserted directly into the inner ear. In many cases, the known ossicular prostheses allow sound transmission between the tympanic membrane and the inner ear only to a limited extent because they can only replace the natural anatomical formations of the ossicular chain in a very limited manner. (See, for example, DE 42 10 235 C1; EP 0 809 982 B1; U.S. Pat. No. 6,387,128 B1 (=reference [2])).

After the prosthesis has been surgically placed in the middle ear and the tympanic membrane is closed again, the so-called healing phase begins. During this time, scars are formed, and they cause unpredictable forces which may result in the prosthesis moving from its local position. In the case of a rigid connection between the head plate and the shaft, increased pressure peaks can occur between the edge of the head plate and the tympanic membrane or the graft between the tympanic membrane and the head plate. These can be so high that penetration or extrusion through the tympanic membrane may result. For this reason, it is very helpful if the prosthesis has a certain post-operative mobility so that the head plate can automatically adapt to the position of the tympanic membrane post-operatively.

Moreover, since the anatomical conditions of the ear, such as the position, the shape and the size of the stirrup bone, the anvil, the hammer and the tympanic membrane vary, it is very advantageous if ossicular prostheses are not designed rigid but have a certain flexibility or variability.

In order to achieve such flexibility/variability, various fastening and coupling devices for ossicles, which have resilient parts and/or joints, are known. Such an articulated connection between a fastening element, mountable on the stirrup foot plate, and the elongate shaft is described for example in EP 1 181 907 B1 (=reference [3]) and is offered by the applicant under the brand name “Ball Joint”.

A further complication, which occurs in various ways, arises due to underventilation of the middle ear space and associated inflammation, tumor formations, adhesive processes in the tympanic membrane region and stiffening of the latter. For example, in the case of a dysfunction of the Eustachian tube, a negative pressure can occur in the middle ear, which can cause a bulge or protrusion (known as retraction) of the tympanic membrane and consequently adhesion for example to the stirrup bone. In order to counteract this and to be able to follow postoperative movements of the tympanic membrane, in known ossicular prostheses, the head plates are designed to be tiltable relative to the connecting element which connects the head plate to the second fastening element and is designed in most cases as an elongate shaft. Such a head plate which is inherently rigid but tiltable relative to the connecting element is described, inter alia, in US 2004/0162614 A1 (=reference [4]).

While reference [3] has an ossicular prosthesis in which the head plate has a built-in ball joint connected to the connecting shaft of the two fastening elements, EP 1 833 424 B1 (=reference [5]) even discloses a connecting shaft designed as a ball chain. As a result, the final axial length of the prosthesis can be set, at least in a stepped manner, by the selection of a certain number of balls in the chain and cutting off the excess balls.

However, a disadvantage of these known ossicular prostheses is that, due to the rigid tilting of the head plate during local medial movements of the tympanic membrane, the opposite side of the head plate is simultaneously moved laterally outwards, whereby pressure peaks are generated on the tympanic membrane.

EP 1 972 307 B1 (=reference [6]) discloses an ossicular prosthesis with a head plate designed to be highly flexible in which, on the one hand, the advantages of the prosthesis according to the above-mentioned reference [5] and also, on the other hand, the advantages of the prostheses described in reference [4] are maintained, but the common disadvantages of a rigid tilting of the head plate are prevented. A problem with the ossicular prosthesis according to reference [6], however, is its limited insertability into the middle ear of the patient because, in particular in the radial direction with respect to its longitudinal axis, the head plate projects significantly laterally and can only be surgically inserted into the middle ear through the tympanic membrane region by means of a large artificial opening. This large opening then of course also grows closed post-operatively with difficulty, and also leaves correspondingly large scars.

The head plate comprises, in reference [6], flexible rib elements which run within the head plate plane and connect a radially outer annular region of the head plate to a central coupling region arranged radially in the center of the head plate. These rib elements, together with the radially outer annular region and the central coupling region, are a fixed component of the head plate disclosed in the reference [6]. They are geometrically designed in such a way that they follow such a medial movement—also locally—during local medial movements of the tympanic membrane, but do not transmit the movement to remote regions of the head plate.

However, the rib elements are not designed for any radial compression of the head plate within the head plate plane and therefore a radial reduction of the head plate diameter, possibly for the purpose of facilitating passage through the tympanic membrane, and would be unsuitable for such a use. This is in particular because the radially outer annular region is rigid and therefore does not follow the attempt of such radial compression or would at most bend uncontrollably.

The ossicular prosthesis described in reference [6] is also entirely unsuitable for a change of length in the direction of the z-axis because, although the connecting element, which extends along the z-axis and rigidly connects the head plate to the second fastening element, is designed to transmit sound, it is designed as such to be rigid. Flexibility of the length of the connecting element in the z-direction is excluded and also not desired at all in the ossicular prosthesis according to reference [6].

EP 3 311 773 B1 (=reference [7]), in contrast, describes an ossicular prosthesis with a head plate that can be folded like an umbrella, and by means of which the prosthesis can be inserted into the middle ear in a virtually minimally invasive manner through a small opening in the tympanic membrane.

Another important issue in the implantation of ossicular prostheses is the setting of the correct axial length of the prosthesis that is optimally adapted to the individual circumstances and geometric conditions in the middle ear of the patient.

Reference [5], discussed above, already proposes a ball chain having separable end balls for this purpose. However, this unfortunately does not allow continuous, but merely a stepwise, length adjustment.

A length-variable ossicular prosthesis having a displacement mechanism, which is installed in the connecting element between the first and second fastening elements, for stepless length adjustment is described in DE 10 2007 041 539 B4 (=reference [8]).

Instead of such a displacement mechanism, which is relatively complex to produce, EP 2 238 946 B1 (=reference [9]) proposes a length-variable ossicular prosthesis in which an accordion-like structure is installed in the connecting element. The axial length of the ossicular prosthesis can then be shortened by an axial compression of this structure and increased by pulling it out.

EP 2 601 909 B1 (=reference [10]) in turn provides a displacement mechanism in the connecting element for the axial length adjustment of the ossicular prosthesis, which displacement mechanism comprises a receiving part and an insertion part which surrounds the receiving part with two legs in a clamp-like manner, the receiving part and the insertion part being displaceable relative to one another in the axial direction of the connecting element.

Finally, a laser-activatable length-variable ossicular prosthesis is disclosed in EP 3 130 315 B1 (=reference [11]). It is proposed therein to construct the connecting element having stretchable and/or compressible partial strands, folded in a loop-like manner, which are made of a shape-memory material. Activation surfaces are connected to the loops of these partial strands in a heat-conducting manner, which surfaces can cause thermal activation, and therefore a deformation of the loops, by the action of heat. In this way, the axial length of the ossicular prosthesis can be changed and adjusted as desired.

Reference [1], already cited above, finally discloses a generic ossicular prosthesis having all of the sets of feature defined at the outset. In particular, it is proposed to provide strip-like rib elements in the connecting element, which rib elements can be spread radially outwards away from the longitudinal axis and in the process shorten the axial length of the connecting element and therefore the axial length of the ossicular prosthesis. The rib elements are spread from the side, i.e. in the radial direction with respect to the connecting element. However, an increase in the axial length is not provided in this mechanism of action. Without additional measures, this length adjustment is also possible only before the prosthesis is inserted into the middle ear of the patient, but not in situ in the implanted state.

### SUMMARY OF THE INVENTION

In contrast, the object of the present invention is to improve a generic length-adjustable ossicular prosthesis of the type defined at the outset using the simplest possible technical means in such a way that a length adjustment in situ of an already placed prosthesis can take place in an uncomplicated and cost-effective manner, that the length adjustment of the prosthesis is also possible from above, that a uniform and symmetrical elongation of the prosthesis is ensured in the process, and that a use of the invention is also made possible, for example, with partial prostheses having a small axial overall height and/or a small functional length.

According to the invention, this object is achieved in a manner that is as surprisingly simple as it is effective in that the rib elements lead at one end directly into coupling regions of the first fastening element within the head plate plane and are movably, but non-detachably, connected thereto, in that all the rib elements are also movably connected to a coupling element and are firmly but detachably connected during operation, the coupling element in turn being rigidly connected at the other end either to the first fastening element or to the second fastening element, in that the rib elements together with the coupling element form the connecting element which connects the two fastening elements to one another in a sound-conducting manner, in that the rib elements are designed such that, in an in-situ state of the ossicular prosthesis inserted in a human middle ear, with the introduction of a force to the rib elements with the force component in parallel with the longitudinal axis in the direction from the first fastening element to the second fastening element, they in each case sectionally assume, in situ, a position located radially further from the longitudinal axis, and therefore shorten, in situ, the axial functional length between the first fastening element and the second fastening element, the rib elements, with the introduction of a force having force components that are anti-parallel with respect to the longitudinal axis, in the direction from the second fastening element to the first fastening element, in each case sectionally assuming, in situ, a position located radially closer to the longitudinal axis, and therefore increasing, in situ, the axial functional length between the first fastening element and the second fastening element, and in that the rib elements retain, in situ, their adjusted radial position relative to the longitudinal axis, when no force acts.

As a result, the advantages of the above-described generic ossicular prosthesis, as described in reference [1], can be used relatively simply, it being possible, however, for the change in length to take place when the prosthesis is already placed within the middle ear, which would not be possible or would be possible only with very significant changes with the prosthesis in reference [1].

Due to the possibility of converting a mechanical movement within the surface of the head plate into an axial length adjustment, the axial movement ultimately always leading out of the head plate plane, with the ossicular prosthesis according to the invention, a finely adjustable length setting can also be carried out only after insertion into the middle ear, i.e. in situ. In this case, the change in length, that is to say the change in the distance between the head plate and the second fastening element at the other end of the prosthesis, is achieved by expansion from the head plate plane downwards (or vice versa).

The mechanism according to the invention is automatically guided within the head plate and therefore ensures uniform and above all symmetrical elongation even without additional measures. In contrast to this, the approaches of the prior art, in particular in reference [1], describe solutions that require the user to manipulate the prosthesis on both sides in order to bring about a symmetrical change in length.

Since the mechanism for length adjustability is integrated in the head plate and is installed with it, the ossicular prosthesis according to the invention can also be produced with a very low overall height.

In addition, the mechanism according to the invention can be operated from above in the head plate. This is also a significant difference from the prior art. For example, the prostheses according to reference [1] enable only a cumbersome manipulation from the side which requires a relatively large amount of space. In contrast, the approach according to the invention has an extremely space-saving design and is also suitable for endoscopic accesses.

Particularly preferred embodiments of the ossicular prosthesis according to the invention are characterized in that the rib elements are designed such that their adjusted radial position relative to the longitudinal axis can be reversibly changed in situ by introducing a corresponding force.

In particular, the movement of the prosthesis parts during the adjustment process can therefore be made reversible, and the prosthesis length can therefore not only be increased, but also shortened again.

The low introduction of force ensures that surrounding middle ear structures are not damaged.

The sophisticated design, according to the invention, of the head plate allows optimum visibility conditions during the surgery.

By means of the lightweight construction, optimal sound transmission is made possible, in particular in the high-frequency range.

As a result of the head plate design according to the invention, optional coupling to the malleus manubrium was made possible.

The design according to the invention was developed such that there are no dead spaces. Therefore, no biofilms can form on the implant and develop implant-associated infections.

Embodiments of the ossicular prosthesis according to the invention in which the coupling element is formed in the shape of a shaft in the direction of the longitudinal axis are also preferred.

Such shaft components can be produced in a manner that is particularly easy and mechanically relatively stable.

These embodiments may advantageously also be improved in that the rib elements are designed such that they may encompass the coupling element like a clamp in a force-fit, or in that the coupling element is designed such that it can engage in the rib elements in a force-fit.

Further advantageous embodiments of the invention are characterized in that locking devices are present which, when an axial force is applied in the direction of the longitudinal axis toward the rib elements and/or at the coupling element, each cause a mechanical resistance at one or more axial lengths of the connecting element.

The adjustment mechanism is therefore provided with locking points in order to achieve a defined axial length of the prosthesis and to fix this length.

The low force application ensures that surrounding middle ear structures are not damaged.

The locking devices enable optimal visibility conditions during the surgery.

The implant can be picked up again during the surgery to ensure a readjustment.

Advantageous developments of these embodiments are characterized in that the coupling element has grooves which extend around the circumference of the shaft in the form of a ring and are arranged at an axial distance from one another at least in a region facing the rib elements during operation, into which grooves radial projections of the rib elements can engage in the implanted state of the ossicular prosthesis, or in that the rib elements have grooves arranged at an axial distance from one another at least in a region facing the coupling element during operation, into which grooves a radial projection of the coupling element can engage in the implanted state of the ossicular prosthesis.

With this groove solution, the rib elements are preferably dimensioned conically such that no play can occur between the elements.

In preferred variants of these developments, the grooves can have defined, in particular identical, axial distances from one another.

As a result, the coupling elements and rib elements can be produced particularly easily. In addition, the final length of the ossicular prosthesis can thereby be adjusted more precisely.

Embodiments are also preferred in which the coupling regions arranged within the head plate plane of the first fastening element are geometrically designed such that they can be used to introduce a force to the rib elements with the force component in parallel or anti-parallel with respect to the longitudinal axis by means of an adjusting tool.

The in-situ length adjustment of the ossicular prosthesis can be achieved by manipulating the structure within the head plate. If the structure is pressed together using an instrument, the prosthesis is extended. If the structure is spread, the length is shortened. In this case, the adjusting tool serves as an implantation aid, and in particular for operating the mechanism for adjusting the length. It can be designed as a minimally invasive, in particular endoscopic, instrument, preferably tweezer-like or pincer-like.

A change in length of the ossicular prosthesis can in principle take place by plastic deformation of the connecting struts and/or by integrated joints.

A first class of embodiments of the ossicular prosthesis according to the invention is characterized in that the rib elements are designed so as to be mechanically rigid at least sectionally, preferably completely.

In this way, a certain flexibility or variability of the prosthesis can be achieved, as is described per se in reference [3]. In view of a particularly high post-operative mobility of the prosthesis, developments can be used in which a plurality of adjacent further rotary elements, preferably a ball joint chain, are used.

In a second class of alternative embodiments of the invention, the rib elements are made of a plastic, flexible material, at least sectionally, the plastic, flexible material of the rib elements having, in particular, an elasticity of ≥1%, preferably an elasticity of ≥2%.

If the rib elements consist of flexible material, the space and the technical effort for the mechanical pivot joints can be saved. If the material of the rib elements has flexibility of >1%, the prosthesis can be constructed particularly “tightly”, it also being ensured that the prosthesis can follow the smallest “topographical” changes in the tympanic membrane.

In a first group of developments of this second class of embodiments, the plastic, flexible material of the rib elements contains highly elastic material, preferably amorphous metal, in particular based on nickel, iron, cobalt or zirconium, and/or a nickel-titanium alloy and/or memory metal.

The above-mentioned properties of flexibility together with good stiffness for sound conduction can be optimally achieved using the mentioned materials.

In particular, the material titanium has, in addition to its strength and excellent sound-transmission properties, also an excellent biocompatibility with the human middle ear as is known. With regard to post-operative position adjustment, embodiments of the invention are advantageous in which the prosthesis or parts thereof, in particular one of the fastening elements, are made of a material having memory effect or superelastic properties, preferably of nitinol, which is known per se for example from EP 1 961 400 B1 (=reference [12]).

In a second group of developments, however, the plastic, flexible material of the rib elements can also contain highly elastic plastic, in particular high-strength elastic polymer, and/or elastic ceramic.

These materials enable ideal preconditions for good sound transmission, with high flexibility and good rigidity, but lower density.

Particularly advantageous are embodiments in which the coupling regions and the rib elements are arranged flat within the head plate plane of the first fastening element before the first introduction of a force to the rib elements with a force component in parallel or anti-parallel with respect to the longitudinal axis.

This enables a particularly compact construction of the ossicular prosthesis according to the invention.

Preferred embodiments of the invention are characterized in that the rib elements extend in a curved and/or meandering and/or zigzag manner between their respective coupling region and the coupling element.

Due to a profile which is not linear but curved instead, the desired effect increases of a local flexibility of the head plate and an only locally limited deviation from smaller medial movements of the tympanic membrane. In addition, the head plate can therefore more easily follow any post-operative change in the tympanic membrane.

In embodiments of the invention, radially outer, free end portions of the rib elements can have atraumatic, non-pointed, free end edges which are radially to the outside with respect to the longitudinal axis and are expanded transversely to the direction of the longitudinal axis, or can be designed as atraumatic surfaces, preferably as closed surfaces or annular surfaces, in particular circular or elliptical. Thus, if a tilting torque arises in the healing phase which results in an increased force effect on the outer end portions, the surface pressure and the risk of extrusion can be reduced.

In further embodiments of the invention, the second fastening element is designed as a plate, in particular as a curved plate, as a sleeve, as a loop, as a closed bell, in particular in the form of a hollow cylinder, as a singly or multiply slotted bell, or as a clip for mechanical connection to a further member of the ossicular chain. Alternative embodiments can provide for the ossicular prosthesis to be coupled directly to the inner ear, at its end supporting the second fastening element, by perforating the stirrup foot plate (= stapedectomy or stapedotomy) and/or by opening the human cochlea (=cochleotomy), in particular by means of a piston.

In addition to the post-operative position shift, there is also a further problem after the implantation of ossicular prostheses, specifically that the middle ear of the human body represents a “half-open bearing.” Each implantation material which is introduced into the body in the context of a reconstruction of the middle ear and its structures, thereby experiences a particular stress due to the fact that a contaminated and infected environment prevails, which usually attacks the material. Since the aim of the implantation of an ossicular prosthesis must also always be to keep the implant in the middle ear of the patient for as long as possible without complications, a prolonged attack on the material can lead to damage to the prosthesis and/or to local infection. Both consequences are not tolerable. In order to permanently prevent damage to both the implantation material and the surrounding tissue, in a further particularly preferred embodiment of the invention, the surface of the ossicular prosthesis is completely or at least sectionally coated with a biologically active coating, in particular a growth-inhibiting and/or a growth-promoting and/or an antibacterial coating. The head plate of the ossicular prosthesis according to the invention should in principle have a growth-promoting coating, while a second fastening element, which leads directly into the inner ear and is designed for example in the form of a piston, has a growth-inhibiting coating.

Alternatively or additionally, in further embodiments, parts of the ossicular prosthesis according to the invention can be made from a ceramic material. However, embodiments of the invention are also possible in which the prosthesis or parts thereof are made of biocompatible plastics, in particular silicone, polytetrafluoroethylene (PTFE) or fiber composite materials. These materials can also prevent post-operative rejection reactions in most cases.

A system comprising an ossicular prosthesis constructed according to the invention of the type described above, and an adjusting tool for manipulating in situ the ossicular prosthesis inserted in the middle ear of a patient, wherein the adjusting tool can also serve as an implantation aid, and which is designed as a minimally invasive, in particular endoscopic, instrument, preferably tweezer-like or pincer-like, is also within the scope of the present invention.

Less trauma in the surgical implantation of the ossicular prosthesis is advantageous here, since only a smaller opening of the tympanic membrane, etc. is necessary.

Finally, a method for implantation of an ossicular prosthesis according to the invention of the type described above, which is characterized in that the first fastening element, in particular the coupling regions within the head plate plane, has structures made of memory metal, and in that a force is applied to the rib elements by heating the structures made of memory metal, preferably in a contactless manner, in particular by light irradiation, for example laser radiation, and the resulting deformation of said memory metal, is also within the scope of the present invention.

In particular, contactless force application into the ossicular prosthesis according to the invention, for example by means of thermal radiation, is therefore made possible.

Further features and advantages of the invention are apparent from the following detailed description of embodiments of the invention, with reference to the figure of the drawing, which show details essential to the invention, and from the claims. The individual features can each be implemented individually or together in any combination in variants of the invention.

### BRIEF DESCRIPTION OF THE DRAWINGS

The schematic drawing shows embodiments of the invention which are explained in greater detail in the following description.

In the drawings:

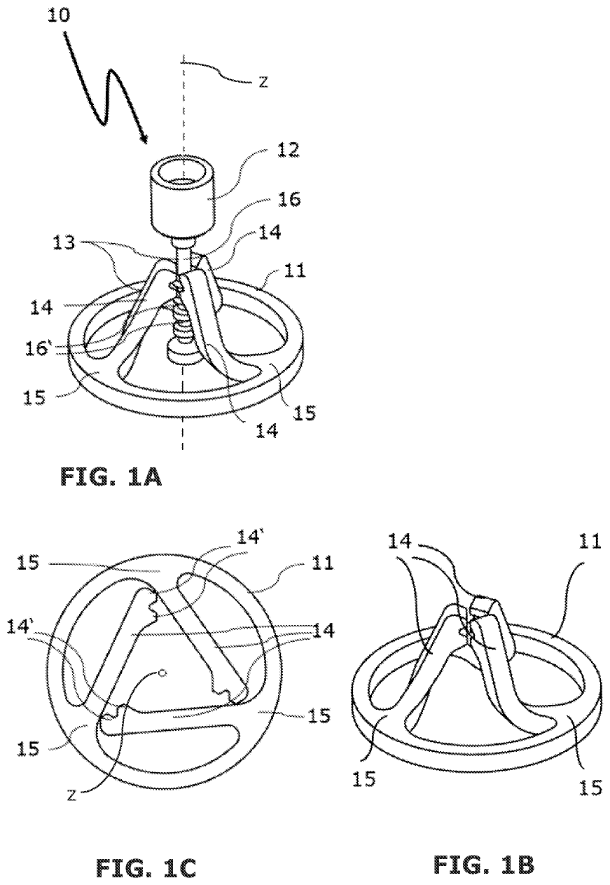

FIGS. 1A-1C show three illustrations of a schematic spatial representation of a first embodiment of the ossicular prosthesis according to the invention, two illustrations, in an oblique view from above, with a viewing direction from the second to the first fastening element, the following being shown in detail:
1A) the complete ossicular prosthesis with a second fastening element designed as a bell open on one side and closed in the radial direction,
1B) the first fastening element of the ossicular prosthesis of FIG. 1A) designed as a head plate with ribs extending out of the head plate plane
1C) a plan view in the z-direction on the head plate plane of the first fastening element of FIG. 1B), but the rib elements being still arranged completely in the head plate plane before an axial force is introduced;

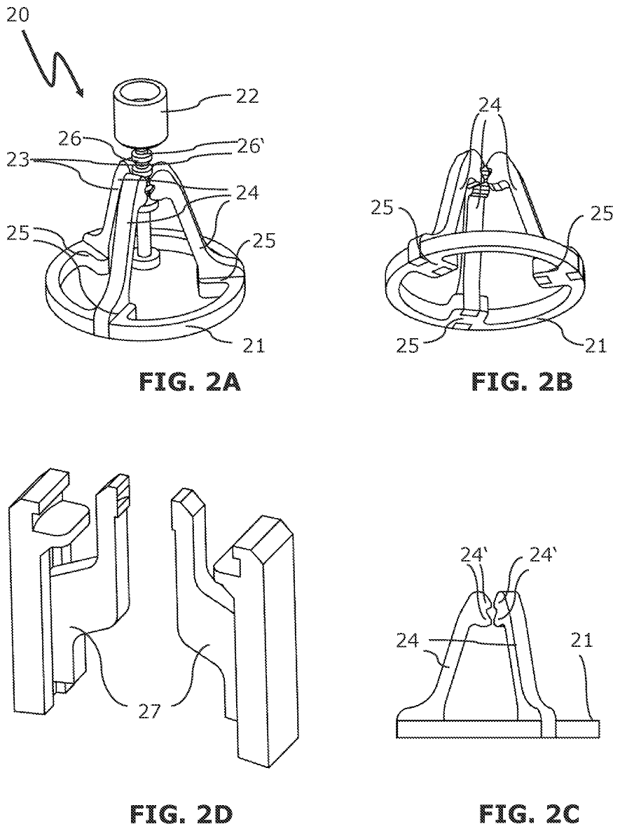

FIGS. 2A-2D shows a further embodiment of an ossicular prosthesis designed according to the invention, specifically:
2A) the complete ossicular prosthesis with a viewing direction obliquely from above from the second to the first fastening element,
2B) the first fastening element of the ossicular prosthesis of FIG. 2A), which is designed as a head plate, with ribs extending out of the head plate plane obliquely from below with a viewing direction of the first fastening element in the direction of the upright ribs,
2C) the head plate with upright ribs of FIG. 2B) with a view from the side perpendicular to the z-direction,
2D) an adjusting tool for manipulation, in particular for the length adjustment of the ossicular prosthesis inserted into the middle ear of a patient;

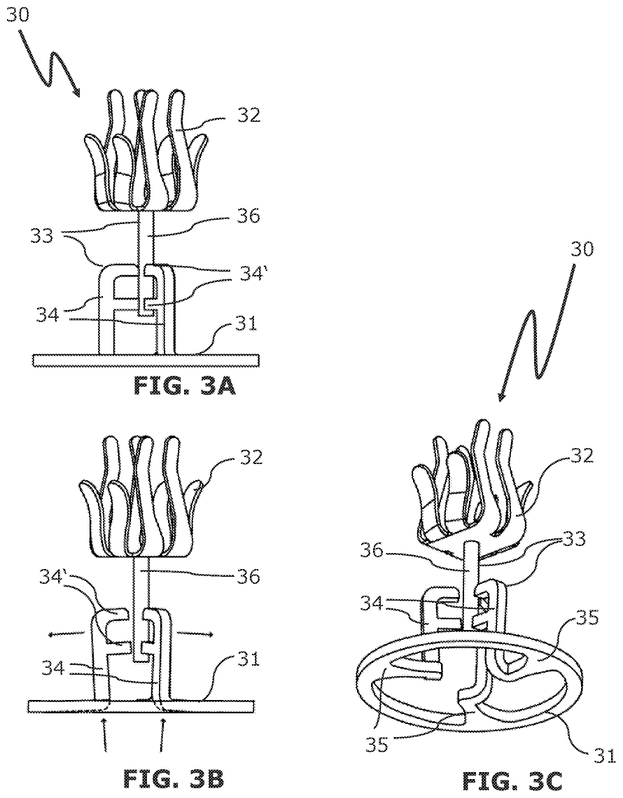

FIGS. 3A-3C show an embodiment of an ossicular prosthesis designed according to the invention, in which the connecting element between the first and second fastening elements comprises a shaft-shaped coupling element without grooves, the second fastening element being designed as a slotted bell, specifically:
3A) the ossicular prosthesis with ribs pressed laterally on the coupling element with a viewing direction from the side perpendicular to the z-direction,
3B) the ossicular prosthesis of FIG. 3A) with ribs raised radially from the coupling element,
3C) the ossicular prosthesis of FIG. 3B) obliquely from below with a viewing direction from the first fastening element in the direction of the second fastening element;

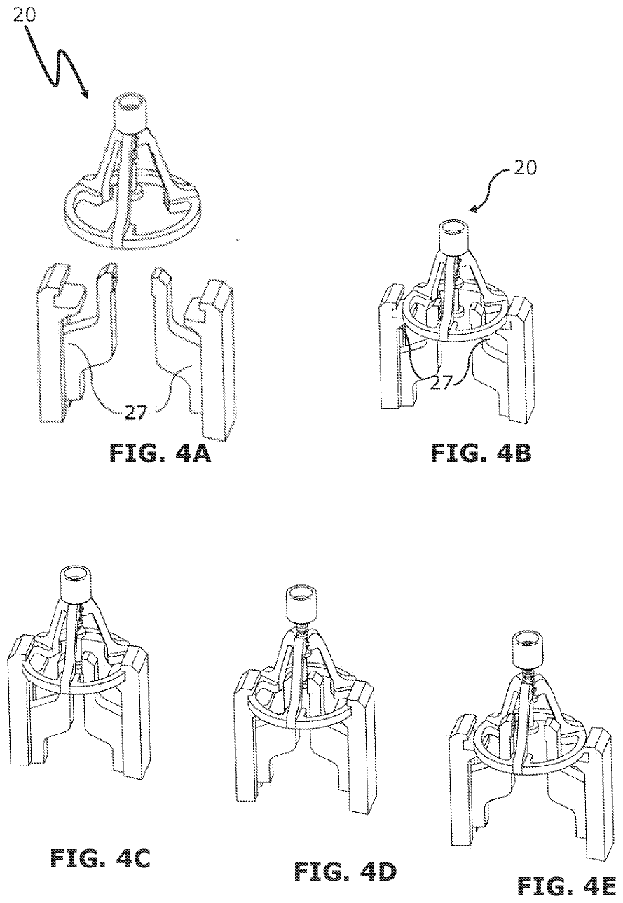

FIGS. 4A-4E show the embodiment of FIG. 2A) in successive stages of the insertion of the adjusting tool from FIG. 2D), specifically:
4A) with the insertion tool at an axial distance in the z-direction from the ossicular prosthesis,
4B) with a head plate of the ossicular prosthesis placed on the insertion tool, the gripping arms of the insertion tool being still radially spaced from the outer contour of the head plate and from the coupling element,
4C) as in FIG. 4B), but with gripping arms of the insertion tool pressed radially on the outer contour of the head plate and on the coupling element,
4D) as in FIG. 4C), but with ribs engaging axially from the second fastening element along the z-axis in the direction of the first fastening element deeper in the coupling element,
4E) as in FIG. 4D), but with gripping arms of the insertion tool that are once again radially released from the outer contour of the head plate and the coupling element; and

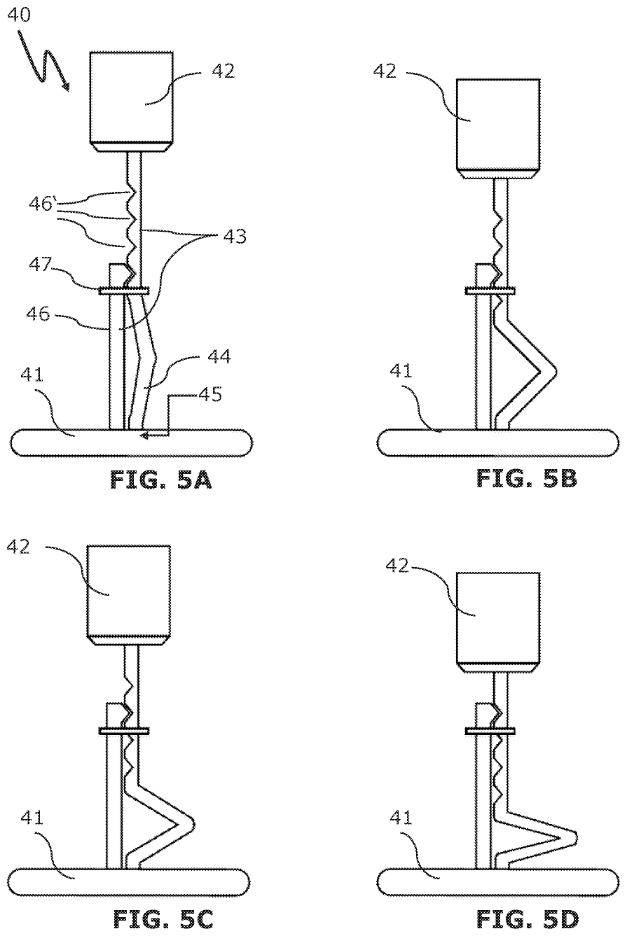

FIGS. 5A-5D show four illustrations of a further embodiment with a viewing direction from the side perpendicular to the z-axis to the ossicular prosthesis according to the invention, the rib element and coupling element of which here are arranged parallel to the z-direction and both open into the first fastening element, the coupling element engaging radially in a force-fitting manner in grooves of the rib element, specifically in four different relative positions of the coupling element and rib, specifically:
5A) with a radial engagement of the coupling element in the groove of the rib element closest to the first fastening element,
5B) as in FIG. 5A), but with a radial engagement of the coupling element in the groove of the rib element second closest to the first fastening element,
5C) as in FIG. 5B), but with a radial engagement of the coupling element in the groove of the rib element second closest to the second fastening element,
5D) as in FIG. 5C), but with a radial engagement of the coupling element in the groove of the rib element closest to the second fastening element.

### DETAILED DESCRIPTION OF THE EMBODIMENTS

The embodiments of the ossicular prosthesis 10; 20; 30; 40 (or parts thereof) according to the invention, shown schematically in the figures of the drawing, each comprise a first fastening element 11; 21; 31; 41 at one end, which is designed in the form of a flat head plate for mechanical contact with the tympanic membrane and/or with the handle of malleus. At the other end of the ossicular prosthesis 10; 20; 30; 40 sits a second fastening element 12; 22; 32; 42 for mechanically connecting the prosthesis to a member or parts of a member of the ossicular chain or to the inner ear. A connecting element 13; 23; 33; 43 which connects the two fastening elements 11, 12 or 21, 22 or 31, 32 or 41, 42 to one another in a sound-conducting manner along a longitudinal axis z, is arranged therebetween.

The connecting element 13; 23; 33; 43 comprises rib elements 14; 24; 34; 44 which, at least sectionally, can be spread, to a greater or lesser extent, radially outwards from the longitudinal axis z, and thereby shorten the axial length of the ossicular prosthesis 10; 20; 30; 40 to a greater or lesser extent.

The invention is distinguished in comparison with known ossicular prostheses of the generic type in that the rib elements 14; 24; 34; 44 at one end directly in coupling regions 15; 25; 35; 45 of the first fastening element 11; 21; 31; 41 within the head plate plane and is movably, but non-detachably, connected thereto,

in that all rib elements 14; 24; 34; 44 with a coupling element 16; 26; 36; 46 are also movable and permanently, but non-detachably connected during operation, the coupling element 16; 26; 36; 46 in turn being rigidly connected at the other end either to the first fastening element 41 or to the second fastening element 12; 22; 32,

in that the rib elements 14; 24; 34; 44 together with the coupling element 16; 26; 36; 46 form the connecting element 13; 23; 33; 43 connecting the two fastening elements 11, 12; 21, 22; 31, 32; 41, 42 to one another in a sound-conducting manner,

in that the rib elements 14; 24; 34; 44 are designed in such a way that, in an in situ state of the ossicular prosthesis 10; 20; 30; 40 inserted in the human middle ear, with the introduction of a force to the rib elements 14; 24; 34; 44 having a force component parallel to the longitudinal axis z in the direction from the first fastening element 11; 21; 31; 41 to the second fastening element 12; 22; 32; 42 in situ, they, in each case sectionally, assume a position lying radially further from the longitudinal axis z and thereby shorten the axial functional length between the first fastening element 11; 21; 31; 41 and the second fastening element 12; 22; 32; 42 in situ, the rib elements 14; 24; 34; 44, with an introduction of a force with a force component anti-parallel to the longitudinal axis z in the direction from the second fastening element 12; 22; 32; 42 to the first fastening element 11; 21; 31; 41 in situ, in each case sectionally, assuming a position lying radially closer to the longitudinal axis z and thereby increasing the axial functional length between the first fastening element 11; 21; 31; 41 and the second fastening element 12; 22; 32; 42 in situ, and

in that the rib elements 14; 24; 34; 44 retain, in situ, their adjusted radial position relative to the longitudinal axis z, when no force acts.

FIGS. 1A) to 1C) show various details of the ossicular prosthesis 10:
FIG. 1A) shows the complete ossicular prosthesis 10 with a second fastening element 12 designed as an axial bell open on one side and closed in the radial direction, and a first fastening element 11 designed as a head plate, from each of which a rib element 14 projects in the z-direction onto the second fastening element 12 over a coupling region 15 and is in mechanical contact in the radial direction with the shaft-shaped coupling element 16 leading into the second fastening element 12.

In this embodiment, the rib elements 14 are designed such that they may encompass the coupling element 16 like a clamp in a force-fit. In its region facing the rib elements 14, the coupling element 16 has axially spaced grooves 16′ running annularly about the shaft circumference, in which radial projections 14′ of the rib elements 14 engage. The projections 14′, together with the grooves 16′, form a locking device which, when an axial force is applied in the direction of the longitudinal axis z to the rib elements 14 and/or to the coupling element 16, each cause a mechanical resistance at one or more axial lengths of the connecting element 13.

FIG. 1A) shows only the first fastening element 11 of the ossicular prosthesis 10, which is designed as a head plate, with rib elements 14 extending out of the head plate plane over the coupling regions 15.

FIG. 1C) shows a plan view in the z-direction of the head plate plane of the first fastening element 11. Here, the rib elements 14 are still arranged completely and flat within the head plate plane. The head plate with the coupling regions 15 and the rib elements 14 can be designed as a one-piece bent sheet metal part.

In FIGS. 2A-2D, a further embodiment of an ossicular prosthesis 20 designed according to the invention is shown.

FIG. 2A) in turn shows the complete ossicular prosthesis 20 with a second fastening element 22, also designed as a closed bell, and a first fastening element 21, designed as a head plate, from each of which a rib element 24 projects in the z-direction onto the second fastening element 22 over a coupling region 25 and is in mechanical contact in the radial direction with the shaft-shaped coupling element 26 leading into the second fastening element 22. Here as well, the rib elements 24 are again designed such that they may encompass the coupling element 26 like a clamp in a force-fit. The coupling element 26 in turn has grooves 26′ extending around the circumference of the shaft and arranged at an axial distance from one another, in which radial projections 24′ of the rib elements 24 engage and, together with the grooves 26′, form a locking device.

FIG. 2B) shows—this time in a spatial view obliquely from below with a viewing direction from the first fastening element 21 in the direction of the upright ribs 24 - in turn only the first fastening element 21 of the ossicular prosthesis 20 designed as a head plate with rib elements 24 extending out of the head plate plane over the coupling regions 25.

FIG. 2C) shows the first fastening element 21 of FIG. 2B) with a view toward it from the side perpendicular to the z-direction. The radial projections 24′ can be seen at the ends of the ribs elements 24 facing away from the head plate.

In FIG. 2D), a spatial-schematic representation of an adjusting tool 27 for in-situ length adjustment of the ossicular prosthesis 20 can be seen, the mode of operation of which is illustrated in detail from the illustrations in FIG. 4.

The length of the ossicular prosthesis 20 is adjusted by means of such instrument in that the rib elements 24 deflect radially away from the z-axis if the shaft 26 is displaced in the axial direction. During assembly, the shaft can be inserted laterally. In this embodiment, the head plate 21 and the rib elements 24 are welded to one another. The shaft with grooves 26′ ensures a defined length adjustment and firm hold.

FIGS. 3A-3C show a further variant of an ossicular prosthesis 30 according to the invention. Here, the connecting element 33 comprises a shaft-shaped coupling element 36 without grooves between the first fastening element 31 and the second fastening element 32 designed as a slotted bell.

FIG. 3A) shows, with a viewing direction from the side perpendicular to the z-direction, the ossicular prosthesis 30 with rib elements 34 pressed laterally against the coupling element 36 via radial projections 34′, while FIG. 3B) shows the ossicular prosthesis 30 of FIG. 3a) in an operating position with ribs elements 34 lifted radially from the coupling element 36 in the direction of the arrow.

FIG. 3C) shows the ossicular prosthesis 30 of FIG. 3B) obliquely from below with a viewing direction from the first fastening element 31 in the direction of the second fastening element 32. Here, it can be clearly seen how the control elements 34 project from the head plate plane of the first fastening element 31 toward the second fastening element 32 over the coupling regions 35.

The three rib elements 34 arranged in a manner distributed around the circumference hold the coupling element 36 by means of a clamping connection. If pressure from below on the head plate 31 causes it to arch over, the rib elements 34 tilt outward and the clamping connection (temporarily) releases. The position of the shaft can then be changed.

FIGS. 4A-4E show the length adjustment of the embodiment of an ossicular prosthesis 20 according to the invention according to FIG. 2A) in successive stages of the insertion of the adjusting tool 27 of FIG. 2d), specifically:
FIG. 4A) shows the ossicular prosthesis 20 with initially maximum axial length in a vertical distance in the z-direction above the adjusting tool 27,
FIG. 4B) shows the ossicular prosthesis 20 with a head plate placed on the insertion tool 27, the gripping arms of the insertion tool 27 being still radially spaced from the outer contour of the head plate and from the coupling element,
FIG. 4C) shows the ossicular prosthesis 20 as in FIG. 4B), but with gripping arms of the insertion tool 27 pressed radially on the outer contour of the head plate and on the coupling element,
FIG. 4D) shows the ossicular prosthesis 20 as in FIG. 4C), but with ribs axially engaging the coupling element lower in the second fastening element along the z-axis in the direction of the first fastening element, i.e. now with a shorter axial length, and
FIG. 4E) shows the shortened ossicular prosthesis 20 as in FIG. 4D), but with gripping arms of the insertion tool 27 that are radially again released from the outer contour of the head plate and from the coupling element.

The two sets of radially inner and outer gripping arms of the instrument can be moved toward one another. As a result, the implant is simultaneously gripped on the head plate and on the shaft. Each arm has a two-part structure, with one part attached to the shaft and the other to the head plate. The two elements are displaceable relative to one another.

As a result, the length of the implant can be adjusted. Ideally, the elements gripping the shaft remain in position relative to the elements in the middle ear. Only the head plate with arms changes its position when the instrument is operated. Thus, the length of the implant changes without exerting pressure on the contacting body parts in the middle ear (stirrup, etc.).

Finally, FIGS. 5A-5D represent, in four different length settings, a further embodiment of the ossicular prosthesis 40 according to the invention, in which the first fastening element 41 is connected to the second fastening element 42 via a rib element 44 which can be bent. The latter is firmly connected to the first fastening element 41 designed as a flat head plate in a coupling region 45, not explicitly shown in the drawing.

At its end facing the second fastening element 42, the rib element 44 has grooves 46′ running annularly around its circumference, in which a radial projection 44′ of the coupling element 46, which runs substantially parallel to the rib element 44 and is firmly anchored in the first fastening element 41, engages radially in a force-fitting manner. Together, the rib element 44 and the coupling element 46 form a connecting element 43.

A ring element 47 is rigidly attached to the coupling element 46, but allows the rib element 44 to pass in the direction of the z-axis through an annular opening (not specifically shown in the drawing). During operation, the ring element 47 thus exerts a radial force on the rib element 44, such that the latter cannot radially avoid the coupling element 46 when sliding axially past it, but always remains nestled against the coupling element 46 during operation.

By introducing a force to the rib element 44 in the direction from the first fastening element 41 to the second fastening element 42, the rib element 44 can be bent laterally, i.e., substantially radially away from the z-axis, thereby shortening the distance between the first fastening element 41 and the second fastening element 42 and thus shortening the axial length of the ossicular prosthesis 40. When force is introduced anti-parallel thereto, the axial length can be increased.

Thus, the axial length of the ossicular prosthesis 40 can be varied within certain limits by varying the degree of lateral bending of the rib element 44. The stability of the structure is achieved by a retainer via the coupling element 46, which is firmly anchored in the first fastening element 41 by means of a latching connection.

FIGS. 5A-5D show four different latching positions of the coupling element 46 and the rib element 44, specifically:
FIG. 5A) with a radial engagement of the coupling element 46 in the groove 46′ of the rib element 44 closest to the first fastening element 41,
FIG. 5B) as in FIG. 5A), but with a radial engagement of the coupling element 46 in the groove 46′ of the rib element 44 second closest to the first fastening element 41,
FIG. 5C) as in FIG. 5B), but with a radial engagement of the coupling element 46 in the groove 46′ of the rib element 44 second closest to the second fastening element 42, and
FIG. 5D) as in FIG. 5C), but with a radial engagement of the coupling element 46 in the groove 46′ of the rib element 44 closest to the second fastening element 42.

Although only a few embodiments of the present invention have been shown and described, it is to be understood that many changes and modifications may be made thereunto without departing from the spirit and scope of the invention.

### LIST OF REFERENCE SIGNS

20; 30; 40 Ossicular prosthesis
11; 21; 31; 41 First fastening element
12; 22; 32; 42 Second fastening element
13; 23; 33; 43 Connecting element
14; 24; 34; 44 Rib elements
14′; 24′; 34′; 44′ Projections
15; 25; 35; 45 Coupling regions
16; 26; 36; 46 Coupling element
16′; 26′; 46′ Grooves
27 Adjusting tool
47 Ring element
z Longitudinal axis

### LIST OF REFERENCES

Publications considered for the assessment of patentability:
[1] U.S. Pat. No. 10,687,937 B2
[2] DE 42 10 235 C1; EP 0 809 982 B1; US 6,387,128 B1
[3] EP 1 181 907 B1
[4] US 2004/0162614 A1
[5] EP 1 833 424 B1
[6] EP 1 972 307 B1
[7] EP 3 311 773 B1
[8] DE 10 2007 041 539 B4
[9] EP 2 238 946 B1
[10] EP 2 601 909 B1
[11] EP 3 130 315 B1
[12] EP 1 961 400 B1

What is claimed is:

1. An ossicular prosthesis (10; 20; 30; 40) which replaces or bridges at least one member or parts of a member of an ossicular chain, the ossicular prosthesis (10; 20; 30; 40) having a first end and second end and comprising:
a first fastening element (11; 21; 31; 41) disposed at the first end and being designed as a flat head plate for mechanical contact with a tympanic membrane and/or with a handle of malleus, and,
a second fastening element (12; 22; 32; 42) disposed at the second end and being configured for mechanical connection to a member or parts of a member of the ossicular chain or to the inner ear, and
a connecting element (13; 23; 33; 43) which connects the two fastening elements (11,12; 21,22; 31,32; 41,42) to one another in a sound-conducting manner along a longitudinal axis (z), and the connecting element (13; 23; 33; 43) having rib elements (14; 24; 34; 44) which—at least sectionally—are configured to be spread to a greater or lesser extent radially outwards away from the longitudinal axis (z), and thereby shorten an axial length of the ossicular prosthesis (10; 20; 30; 40) to a greater or lesser extent,
wherein
the rib elements (14; 24; 34; 44) lead at one end directly into coupling regions (15; 25; 35; 45) of the first fastening element (11; 21; 31; 41) within a head plate plane and are movably, but non-detachably, connected thereto,
all of the rib elements (14; 24; 34; 44) are also movably connected to a coupling element (16; 26; 36; 46) and are firmly but detachably connected during operation, the coupling element (16; 26; 36; 46) in turn being rigidly connected at another end either to the first fastening element (41) or to the second fastening element (12; 22; 32), wherein the rib elements (14; 24; 34; 44) together with the coupling element (16; 26; 36; 46) form the connecting element (13; 23; 33; 43) which connects the two fastening elements (11, 12; 21, 22; 31, 32; 41, 42) to one another in a sound-conducting manner,
the rib elements (14; 24; 34; 44) are designed such that, in an in situ state of the ossicular prosthesis (10; 20; 30; 40) inserted in a human middle ear, with the introduction of a force to the rib elements (14; 24; 34; 44) with a force component in parallel to a longitudinal axis (z) in a direction from the first fastening element (11; 21; 31; 41) to the second fastening element (12; 22; 32; 42), the rib elements in each case sectionally assume, in situ, a position located radially further from the longitudinal axis (z) and therefore shorten, in situ, an axial functional length between the first fastening element (11; 21; 31; 41) and the second fastening element (12; 22; 32; 42), the rib elements (14; 24; 34; 44), with the introduction of a force having force components that are anti-parallel with respect to the longitudinal axis (z), in the direction of the second fastening element (12; 22; 32; 42) to the first fastening element (11; 21; 31; 41), in each case sectionally assuming, in situ, a position located radially closer to the longitudinal axis (z), and therefore increasing, in situ, the axial functional length between the first fastening element (11; 21; 31; 41) and the second fastening element (12; 22; 32; 42), and
the rib elements (14; 24; 34; 44) retain, in situ, their adjusted radial position relative to the longitudinal axis (z), when no force acts.

2. The ossicular prosthesis according to claim 1, wherein the rib elements (14; 24; 34; 44) are designed such that their adjusted radial position relative to the longitudinal axis (z) can be reversibly changed in situ by introducing a corresponding force.

3. The ossicular prosthesis according to claim 1, wherein the coupling element (16; 26; 36; 46) is formed in the shape of a shaft in the direction of the longitudinal axis (z).

4. The ossicular prosthesis according to claim 3, wherein the rib elements (14; 24; 34; 44) are designed so as to encompass the coupling element (16; 26; 36) like a clamp in a force-fit, or
wherein the coupling element (46) is designed such that the coupling element can engage in the rib elements (44) in a force-fit.

5. The ossicular prosthesis according to claim 1, further comprising locking devices that are configured such that when an axial force is applied in the direction of the longitudinal axis (z) to the rib elements (14; 24; 34; 44) and/or at the coupling element (16; 26; 36; 46), each locking device causes a mechanical resistance at one or more axial lengths of the connecting element (13; 23; 33; 43).

6. The ossicular prosthesis according to claim 5, wherein the coupling element (16; 26; 36) is formed in the shape of a shaft that has grooves (16′; 26′), which extend around the circumference of the shaft in the form of a ring and are arranged at an axial distance from one another at least in a region facing the rib elements (14; 24; 34) during operation, into which grooves radial projections (14′; 24′; 34′) of the rib elements (14; 24; 34) can engage in an implanted state of the ossicular prosthesis (10; 20; 30), or
wherein the rib elements (44) have grooves (46′) arranged at an axial distance from one another at least in a region facing the coupling element (46) during operation, into which grooves a radial projection (44′) of the coupling element (46) can engage in the implanted state of the ossicular prosthesis (40).

7. The ossicular prosthesis according to claim 6, wherein the grooves (16′; 26′; 46′) have defined, identical axial distances from one another.

8. The ossicular prosthesis according to claim 1, wherein the coupling regions (15; 25; 35; 45) arranged within the head plate plane of the first fastening element (11; 21; 31; 41) are geometrically designed such that the coupling regions can be used to introduce a force to the rib elements (14; 24; 34; 44) with a force component in parallel or anti-parallel with respect to the longitudinal axis (z) by means of an adjusting tool (27) in situ.

9. The ossicular prosthesis according to claim 1, wherein the rib elements (14; 24; 34; 44) are designed so as to be mechanically rigid at least sectionally.

10. The ossicular prosthesis according claim 1, wherein the rib elements (14; 24; 34; 44) are made of a plastic, flexible material, at least sectionally, and wherein the plastic, flexible material of the rib elements (14; 24; 34; 44) has, in particular, an elasticity of ≥1%), preferably an elasticity of ≥2%.

11. The ossicular prosthesis according to claim 10, wherein the plastic, flexible material of the rib elements (14; 24; 34; 44) contains highly elastic material, preferably amorphous metal, in particular based on nickel, iron, cobalt or zirconium, and/or a nickel-titanium alloy and/or memory metal.

12. The ossicular prosthesis according to claim 10, wherein the plastic, flexible material of the rib elements (14; 24; 34; 44) contains a highly elastic plastic, in particular a high-strength elastic polymer, and/or elastic ceramic.

13. The ossicular prosthesis according to claim 1, wherein the coupling regions (15; 25; 35; 45) and the rib elements (14; 24; 34; 44) are arranged flat within the head plate plane of the first fastening element (11; 21; 31; 41) before the first introduction of a force to the rib elements (14; 24; 34; 44) with a force component in parallel or anti-parallel with respect to the longitudinal axis (z).

14. The ossicular prosthesis according to claim 1, wherein the rib elements (14; 24; 34; 44) extend between their respective coupling region (15; 25; 35; 45) and the coupling element (16; 26; 36; 46) in a curved and/or meandering and/or zigzag manner.

15. A system comprising an ossicular prosthesis (10; 20; 30; 40) according to claim 8 and an adjusting tool (27) for manipulating in situ the ossicular prosthesis (10; 20; 30; 40. inserted in the middle ear of a patient, wherein the adjusting tool (27) is designed as a minimally invasive, in particular endoscopic, instrument.

16. The system according to claim 15, wherein the adjusting tool is in form of pincers or tweezers.
